(19) United States
(12) Patent Application Publication
(10) Pub. No.: US 2008/0125400 A1
(43) Pub. Date: May 29, 2008

(54) USE OF PHOSPHOENOLPYRUVATE DERIVATIVES

(76) Inventor: Christopher Speirs, Leatherhead (GB)

Correspondence Address: CONLEY ROSE, P.C.;David A. Rose, P. O. BOX 3267, HOUSTON, TX, 77253-3267, US

(21) Appl. No.: 11/563,388

(22) Filed: Nov. 27, 2006

(51) Int. Cl. A61K 31/66

(52) U.S. Cl. 514/109

(57) ABSTRACT

A therapeutically effective amount of at least one phosphoenolpyruvate derivative having Formula (1):

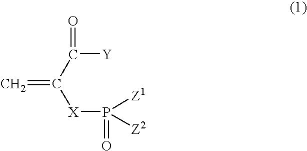

wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
   and wherein:
R¹ to R⁶ are selected, independently, from the group consisting of H; alkyl; cycloalkyl; alkenyl; aryl; and aralkyl,
   or a physiologically acceptable salt thereof is administered to a patient to reduce or inhibit necrosis of cardiac muscle tissue as evidenced by a reduction in the levels of cardiac troponins in the blood. A particularly preferred derivative is phosphoenolpyruvic amide (“PEPA”), i.e. wherein X═O; Y═NH₂; and Z¹=Z²=OH.

### REFERENCE TO RELATED APPLICATIONS

Not Applicable.

### STATEMENT REGARDING FEDERALLY SPONSORED RESEARCH OR DEVELOPMENT

Not Applicable.

### TECHNICAL FIELD OF THE INVENTION

Embodiments of the invention relates to a novel use of certain derivatives, particularly amide derivatives, of phosphoenolpyruvate (“PEP”). In particular, the embodiments can be used to reduce or inhibit necrosis of cardiac muscle tissue.

### BACKGROUND OF THE INVENTION

Necrosis is the death of cells or tissue through injury or disease. Necrosis may be caused by ischemia which refers to an insufficient supply of blood to an organ, usually due to a blockage in an artery caused by a build up of atherosclerotic plaque. Ischemia of cardiac muscle tissue is observed when a patient undergoes cardiac bypass surgery or suffers from acute coronary syndrome (“ACS”).

ACS is a general term covering any group of clinical symptoms compatible with acute myocardial ischemia. Myocardial ischemia is an intermediate condition in coronary artery disease during which cardiac muscle tissue is slowly or suddenly starved of oxygen and other nutrients. If blood flow to the myocardium is reduced to below a particular threshold, ischemia can lead to myocardial infarction. Therefore, a spectrum of clinical conditions, ranging from unstable angina to non-Q-wave and Q-wave myocardial infarctions, is covered by ACS. Life-threatening disorders such as these are a major cause of emergency medical care and hospitalization, particularly in the USA.

The extent of necrosis of cardiac muscle tissue has a significant impact not only on the survival rate of patients undergoing cardiac surgery or suffering from ACS but also on the probability that surgical intervention (or further surgical intervention) is required at some point in the future. Thus, there is a need to develop a treatment which reduces necrosis of the myocardium observed in these patients. Similarly, there is also a need to develop a treatment for patients at high risk of myocardial necrosis such that necrosis of the cardiac muscle tissue is inhibited.

Necrosis may be identified by the level of troponin in the blood. Troponin is a protein complex found in muscle cells that responds to changes in the intracellular concentration of calcium, thereby enabling contraction and relaxation of muscles within the body. Troponin has three subunits; troponin C (“TnC”), troponin I (“TnI”) and troponin T (“TnT”).

These subunits are usually released into the blood only when muscle cells are damaged or die. Accordingly, the normal concentration in the blood of troponin is very low, reflecting the normal rate of apoptosis (or programmed cell death) of muscle cells in the body. For example, the concentration of TnI is usually below about 1.6 nanograms per milliliter (“ng/mL”) and the concentration of TnT is usually below about 0.1 ng/mL. However, when muscle cells are damaged or are undergoing active necrosis, the amount of troponin subunits in the blood increases significantly.

Cardiac TnI and TnT are very sensitive and specific indicators of damaged and necrosing heart tissue. The concentrations of these troponin subunits are measured in the blood of patients with chest pain to differentiate between unstable angina and myocardial infarction. For example, a patient who has suffered a recent myocardial infarction would have an area of damaged or necrosing heart muscle and so would have elevated concentrations of cardiac TnI and TnT in the blood. The amounts of TnI and TnT are conventionally measured by immunoassay methods. For example, troponin levels may be measured using the ADVIA Centaurs System (Bayer HealthCare LLC Diagnostics Division, Tarrytown, N.Y. 10591, USA).

Treatment of ACS includes interventions to unblock an occluded artery and to revascularize the heart. In cardiac surgery, the heart is bypassed using a heart-lung bypass machine and is stopped by perfusing a cardioplegic solution to the heart. It is common practice to add components to cardioplegic solutions which, during surgery, inhibit damage to the heart tissue due to hypoxia and which aid recovery of cardiac function when the heart is restarted. Water soluble salts of both PEP and adenosine triphosphate (“ATP”) have been used as such additives.

PEP is a glycolytic substrate which combines with adenosine diphosphate (“ADP”) to form pyruvate and ATP. The exergonic reaction is catalysed by pyruvate kinase and is irreversible under intracellular conditions, requiring Mg²⁺ as a cofactor and an alkali metal cation (e.g. K⁺) as a physiological activator. The enzyme is activated by increases in glycolytic intermediates such as fructose-1,6-bisphosphate or PEP, or by low ATP concentrations, and is inhibited by high ATP concentrations or when aerobic metabolites such as fatty acids or acetyl CoA are available.

WO 83/02391 A1 (Hultman et al) discloses a pharmaceutical composition comprising water soluble salts of both PEP and ATP to prevent and treat ischaemic cell damage following parenteral administration. Areas of application for the PEP/ATP composition are disclosed as a perfusion and preservation solution for use in open heart surgery and other organ transplants, and for treating ischaemic brain and heart damage as a result of heart failure, drowning or drug overdose.

It is disclosed in WO 98/22479 A1 (Dogadina et al) that certain derivatives of PEP, in particular phosphoenolpyruvic amide (“PEPA”), can also be used in cardioplegia and solutions for perfusion and reperfusion to aid recovery of function of ischaemic tissue during and after open heart surgery.

### SUMMARY OF EMBODIMENTS OF THE INVENTION

The Inventor has discovered that certain derivatives of PEP unexpectedly reduce the level of cardiac troponin subunits in the blood of patients undergoing cardiac surgery on cardiopulmonary bypass. Such a reduction in the level of cardiac troponin subunits is indicative of a corresponding reduction of necrosis of cardiac muscle tissue.

According to a first aspect of an embodiment of the invention, there is provided a method of reducing or inhibiting necrosis of cardiac muscle tissue in a patient comprising administering to said patient a therapeutically effective amount of at least one compound having Formula (1):

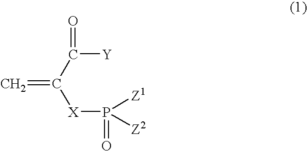

wherein:

X is selected from the group consisting of O; and NH;

Y is selected from the group consisting of OH; OR¹; and NR²R³;

Z¹ and Z² are selected, independently, from the group consisting of OH; OR⁴; and NR⁵R⁶;

and wherein:

R¹ to R⁶ are selected, independently, from the group consisting of H; alkyl; cycloalkyl; alkenyl; aryl; and aralkyl,

or a physiologically acceptable salt thereof.

Surgical intervention may result in myocardial ischemia. For example, where the intervention is cardiac bypass surgery, blood flow through the myocardium is stopped and, thus, cardiac muscle tissue is starved of oxygen and other nutrients. In addition, in other forms of intervention, e.g. percutaneous angioplasty where blood flow is not stopped, necrosis of cardiac muscle tissue might occur if the tissue suffers trauma and is damaged. Thus, according to a second aspect of an embodiment of the invention, there is provided a method of reducing necrosis of cardiac muscle tissue in a patient undergoing cardiac surgery comprising administering to said patient a therapeutically effective amount of at least one of said compounds or salts.

Embodiments of the invention may also be applied to patients suffering from a cardiac event or having suffered a recent cardiac event, resulting in necrosis of cardiac muscle tissue. Cardiac events would include myocardial infarctions and angina. These patients are referred to in this application as suffering from ACS. Thus, according to a third aspect of an embodiment of the invention, there is provided a method of treatment of a patient suffering from ACS comprising administering to said patient a therapeutically effective amount of at least one of said compounds or salts.

The prevention of necrosis in a patient at high risk of myocardial necrosis is also an important aspect of an embodiment of the invention. Thus, according to a fourth aspect, there is provided a method of inhibiting necrosis of cardiac muscle tissue in a patient at high risk of cardiac muscle necrosis comprising administering to said patient a therapeutically effective amount of at least one of said compounds or salts.

There is provided, in a fifth aspect, a method of limiting troponin levels in blood of a patient comprising administering to said patient a therapeutically effective amount of at least one of said compounds or salts.

### BRIEF DESCRIPTION OF THE DRAWINGS

FIG. 1 is a graphical representation of the mean (±SEM) change in concentration of TnI in micrograms per liter (“mcg/L” or “μg/L”) over time observed in the study exemplified below.

### DETAILED DESCRIPTION OF EMBODIMENTS OF THE INVENTION

Embodiments of the invention comprise a method of reducing or inhibiting necrosis of cardiac muscle tissue in a patient comprising administering to said patient a therapeutically effective amount of at least one compound having Formula (1):

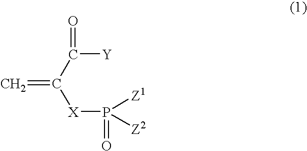

wherein:

X is selected from the group consisting of O; and NH;

Y is selected from the group consisting of OH; OR¹; and NR²R³;

Z¹ and Z² are selected, independently, from the group consisting of OH; OR⁴; and NR⁵R⁶;

and wherein:

R¹ to R⁶ are selected, independently, from the group consisting of H; alkyl; cycloalkyl; alkenyl; aryl; and aralkyl,

or a physiologically acceptable salt thereof.

The expression “reducing or inhibiting necrosis” is intended to mean that the or each compound lessens, hinders or prevents necrosis of cardiac muscle tissue. Without wishing to be bound by any particular theory, the Inventor currently believes that necrosis is reduced or inhibited by a substrate effect in which the high energy phosphate ATP is generated during the metabolism of the compound, or the compound exerts an antioxidant effect.

The therapeutic amount of said at least one compound having Formula (1) usually depends on the mode of administration. However, the therapeutic amount is usually sufficient to provide a reduction in peak change in troponin concentration in blood of a patient of at least about 15%, preferably at least about 40%, and most preferably at least about 50%, of the expected peak change in troponin concentration in blood.

Examples of suitable therapeutic amounts for enteral and parenteral administration are usually found within the range of about 0.01 g to about 100 g. Daily doses may be given as single bolus injections, divided bolus injections or continuous infusions. For intravenous administration, examples of therapeutic amounts are usually found within the range of about 0.1 g to about 10 g for single doses and within the range of about 0.01 g to about 10 g for repeat doses. A single dose is usually not more than about 10 g and preferably not more than about 2.1 g. A suitable single dose may be from about 0.01 g to about 2 g, e.g. about 0.7 g. For other routes of administration, e.g. oral, a suitable dosage may be between about 0.01 g to about 100 g depending, for example, on the level of absorption.

The actual therapeutic amount used to treat a patient and the mode of administration is to be determined by a qualified medical practitioner. However, the total dose is usually not less than 0.01 g and usually not more than 5 g per day.

The term “alkyl” refers to straight and branched chain alkyl groups usually containing from 1 to 6 carbon atoms, preferably 1 to 4 carbon atoms. Suitable examples include methyl; ethyl; propyl; isopropyl; butyl; sec-butyl; tert-butyl; pentyl; and hexyl groups.

The term “cycloalkyl” refers to saturated hydrocarbon rings, preferably having up to 7 carbon atoms. Suitable examples include cyclopentyl; and cyclohexyl groups.

The term “alkenyl” refers to straight or branched chain, unsaturated or partially saturated, hydrocarbon groups, preferably containing from 2 to 7 carbon atoms. Suitable examples include ethylenyl; propylenyl; and butylenyl groups.

The term “aryl” refers to a benzenoid aromatic group, preferably phenyl.

The term “aralkyl” refers to an aryl group substituted with one or more alkyl groups.

The expression “physiologically acceptable salts” is intended to mean salts of the compounds having Formula (1) which not only reduce or inhibit necrosis of cardiac muscle tissue but also which do not cause an adverse side effect within the body or cause only tolerable side effects. Suitable salts are converted within the body to the same active metabolite as that for the compound having Formula (1).

Preferred salts may be derived from an appropriate base, such as an alkali metal (for example, sodium; and potassium); an alkaline earth metal (for example, magnesium); ammonium and NX₄⁺ (wherein X is C₁₋₄ alkyl).

Physiologically acceptable salts of an amino group include salts of organic carboxylic acids (such as acetic acid; lacetic acid; tartaric acid; malic acid; isethionic acid; lactobionic acid; and succinic acid), organic sulphonic acids (such as methanesulphonic acid; ethanesulphonic acid; benzenesulphonic acid and p-toluenesulphonic acid) and inorganic acids (such as hydrochloric acid; sulphuric acid; phosphoric acid; and sulphamic acid).

Physiologically acceptable salts of a compound of a hydroxy group include the anion of said compound in combination with a suitable cation such as NH₄⁺ and NX₄⁺ (wherein X is a C₁₋₄ alkyl group).

Preferred PEP derivatives include those derivatives having Formula (1) in which X is O; Y is NR²R³; Z¹ is OH; and Z² is OH. A particularly preferred PEP derivative is phosphoenolpyruvic amide (“PEPA”) having Formula (1) in which X is O; Y is NH₂; and Z¹=Z²=OH. The IUPAC name for the particularly preferred compound is 2-(dihydroxyphosphoryloxy)prop-2-enoic amide.

In embodiments using a salt of the compounds of Formula (1) wherein X═O; Y═NR²R³; and Z¹=Z²=OH, preferred salts include alkali metal salts, particularly potassium metal salts.

Any suitable method may be used to produce the compounds having Formula (1) or the salts thereof. However, a preferred method is disclosed in WO 98/22479 A1, the disclosure of which is incorporated herein by reference.

Where more than one compound is administered, the compounds may be administered simultaneously or sequentially. However, in such embodiments, the compounds are preferably administered simultaneously.

Studies indicate that the compounds of Formula (1) and their salts have a “protective” effect on cardiac muscle tissue. Thus, embodiments of the invention are suitable for application to patients undergoing a surgical intervention. Examples of surgical intervention include percutaneous angioplasty. Embodiments of the invention have particular application to patients undergoing cardiac surgery on cardiopulmonary bypass, usually following a cardiac event associated with ACS. Preferably, in these embodiments, the or each compound or salt is administered in the form of an additive to a cardioplegic solution.

Studies indicate that, during and following cardiac surgery, the observed level of necrosis of cardiac muscle tissue at a given point within 72 hours after administration of a compound according to Formula (1) is significantly less than the expected level of necrosis at said point if said compound(s) had not been administered. The reduction of the level of necrosis relative to the expected level is usually at least about 15%, preferably at least about 40% and most preferably at least about 50%. This reduction may be up to about 60%.

Embodiments of the invention are also suitable to treat clinical indications identified by EKG changes associated with necrosis of cardiac muscle tissue or high risk of cardiac necrosis. Indications that may be treated using the present method include ACS (e.g. myocardial infarction including presumptive diagnoses and patients at high risk of imminent infarction including various types of angina such as unstable angina, variant angina and stable angina. Unstable angina can be defined by non-ST-segment angina, whereas variant angina has ST-segment elevation during an attack and stable angina can be defined by ST-segment elevation of the EKG.

Patients at “high risk” may have had a recent infarction or may display clinical symptoms, EKG and/or have biochemical findings suggestive of an imminent myocardial infarction or a myocardial infarction in progression. This group of patients may also include those patients with poor myocardial function (e.g. low EF %).

If the compounds or salts of embodiments of the invention are administered after a recent cardiac event, then the or each compound or salt is preferably administered as soon as possible, e.g. within a few minutes, after diagnosis of the condition and diagnosis should be made as soon as possible after the event.

The or each compound or salt is preferably administered no more than about 24 hours after the cardiac event, more preferably within about 6 hours after the event, and most preferably within about 1 hour after the event. The or each compound or salt is preferably administered as soon as possible after the onset of a necrotizing process or when there are symptoms and signs suggestive of a high risk of the onset of cardiac muscle ischemic necrosis.

If the compounds or salts of embodiments of the invention are administered before the onset of necrosis, then the or each compound or salt is preferably administered as soon as possible (e.g. within about 24 hours, preferably within about 6 hours and most preferably with about 1 hour) after diagnosis of the condition. Again, the diagnosis should be made as soon as possible.

The or each compound or salt may be administered to patients having poor cardiac function, by which is meant that the cardiac function is lower than normal. However, the or each compound or salt may be administered to a patient having normal cardiac function or whose cardiac function is not significantly reduced.

The expression “normal cardiac function” is intended to mean that the cardiac function of the patient is within normal parameters as would be assessed by a qualified cardiologist. For example, the characteristics of “normal cardiac function” would include a left ventricle ejection fraction (“EF”) of at least 55%. The characteristics of “poor” cardiac function would include an EF of less than 50%, e.g. from about 25% to about 40%.

The formulations for administration of the compounds of Formula (1) or their salts will depend on the application of the invention. For example, if a compound is to be administered as soon as possible after a recent cardiac event, then the formulation ideally should be able to deliver the compound(s) to the required site quickly and efficiently.

The formulations of the compounds of the invention include those suitable for oral; parenteral (including subcutaneous; intradermal; intramuscular; intravenous; and intraarticular); rectal; and topical (including dermal; buccal; sublingual; and intraocular) administration although the most suitable route may depend upon for example the condition and disorder of the recipient.

Preferred formulations are usually solutions suitable for intravenous administration (by injection or drip) or for perfusion or reperfusion during surgery. In addition to at least one compound of the invention, such a solution is usually aqueous and may comprise compounds of sodium; potassium; calcium; magnesium; and buffering agents. More specifically, a solution may comprise (in addition to a compound of the invention) sodium chloride; magnesium chloride; potassium chloride; calcium chloride; potassium bicarbonate; and potassium biphosphate.

Formulations for parenteral administration include aqueous and non-aqueous sterile injection solutions which may contain anti-oxidants; buffers; bacteriostats; and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets.

A suitable formulation for administration during cardiac surgery is a cardioplegic solution for perfusion to the heart. The PEP derivative(s) or salt(s) may be dissolved in Ringer's solution in a concentration of from about 50 micromoles per liter (“μM/L”) to about 5000 μM/L and preferably from about 100 μM/L to about 1000 μM/L.

The PEP/Ringer's solution may then be mixed as needed to produce the defined cardioplegia composition which, for a ratio of 1:1 blood:crystalloid), would be to add two 20 ml ampoules of concentrate to each liter of Ringer's solution for dilution 1:1 with blood from the cardiopulmonary bypass machine| with a cardioplegic solution concentrate such as the normal St. Thomas' Hospital cardioplegic solution (“STH”) concentrate (Martindale Pharmaceuticals Ltd., Brentwood, Essex, CM14 4LZ). STH is currently marketed in the US under the trade mark PLEGISOL™.

The mixture is then usually mixed with pump blood in theatre at a suitable ratio of mixture to blood. This ratio may be about 2:1 to about 1:2, preferably 1:1|. However, some surgeons, particularly in US, would use a more normal mixture of 4:1 (blood:crystalloid) or even up to 8:1 (and these mixtures would obviously require more of the concentrate.

Formulations of embodiments of the invention suitable for oral administration may be presented as discrete units such as capsules sachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a base such as gelatin and glycerin or sucrose and acacia. Quick delivery of the compound(s) or salt(s) may be achieved using a sublingual spray.

The compounds of the invention may be administered via injection or orally at a dose range for adult humans of from 1 g to 25 g/day, such as about 15 g/day. Tablets or other forms of presentation provided in discrete units may conveniently contain an amount of compound of the invention which is effective at such dosage or as a multiple of the same, for example, units containing 500 mg, 1 g or 2 g.

The formulations may conveniently be presented in unit dosage form and may be prepared by any of the methods well known in the art of pharmacy. All methods include the step of bringing into association a compound of formula (I) and all salts, esters, amides and physiologically acceptable prodrugs thereof (“active ingredient”) with the carrier which constitutes one or more accessory ingredients. In general the formulations are prepared by uniformly and intimately bringing into association the active ingredient with liquid carriers or finely divided solid carriers or both, and then, if necessary, shaping the product into the desired formulation.

Troponin levels in blood of a patient are limited by administering to said patient a therapeutically effective amount of at least one compound having Formula (1):

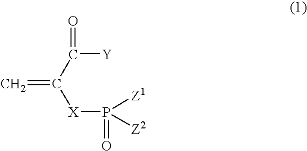

wherein X, Y, Z¹ and Z² are as defined above,
or a physiologically acceptable salt thereof.

The therapeutic amount usually provides a reduction in peak change in troponin (e.g. TnI) concentration in blood of a patient of at least about 15%, preferably at least about 40%, and most preferably at least about 50%, of the expected peak change in troponin (e.g. TnI) concentration in blood. The therapeutic amount may limit the peak change of TnI concentration in blood to no more than about 7 μg/L.

### EXAMPLE

A study was carried out to determine the effect of PEPA on heart tissue in patients with ischemic heart disease undergoing coronary artery bypass surgery. The study was a randomized, double-blind, parallel group, comparative trial that compared the effect during surgery (and shortly thereafter) of the presence or absence of a single dose of PEPA in blood-based STH.

The patients participating in the study had an ejection fraction of from 25% to 40% and needed coronary artery bypass surgery. The patients were assigned randomly to one of two groups. For the first group, PEPA was made up in a concentration of 495 μM/L as a single dose added to blood-based STH (“PEPA+STH”). Normal blood-based STH (without PEPA) was administered to the second group. There were 37 patients in each group.

PEPA was made up under sterile conditions in 40 ml of Ringer's solution and then filtered using a sterile 0.22 μm filter to sterilize the solution. Ten mL aliquots of the sterile PEPA solution were measured and added to each of three 1 L bags of Ringer's solution under sterile conditions providing a Ringer's solution containing 990 μM/L PEPA. Three 1 L bags of PEPA/Ringer's solution were made up for each patient in the PEPA+STH group and sent to theatre just before surgery. For patients in the non-PEPA group, three 1 L bags of Ringer's solution were labeled and sent to theatre before surgery in the usual way.

In theatre, the normal St Thomas' Hospital cardioplegic solution concentrate was added to make a double-strength cardioplegia in the Ringer's solution or PEPA/Ringer's solution before mixing 1:1 with pump blood to make the blood-based cardioplegic solution (with PEPA at 495 μM/L in the PEPA-STH solution). Each patient's heart was perfused with an appropriate volume (usually 1000 ml) at the onset of the isehaemic period during the operation with one of the two resultant cardioplegic solutions, i.e. with or without PEPA, and subsequently with further appropriate volumes (usually 300-500 ml) at appropriate durations of ischaemia (usually every 30 minutes). Perfusion took place at the normal time for coronary artery bypass operations after the chest was opened.

A sample of blood was collected from each patient at regular intervals during surgery (at the time of anaesthesia and at 6 and 12 hours after the start of surgery) and after surgery (at 24, 48 and 72 hours after the start of surgery). Each sample was analyzed by an immunoassay method using an ADVIA Centaur® system to determine the level of troponin I.

The results of the immunoassays were subjected to statistical analysis using SASS statistical software (release 6.12). All significance tests were two-tailed and carried out at the 5% level. No corrections for multiple statistical significance testing were performed.

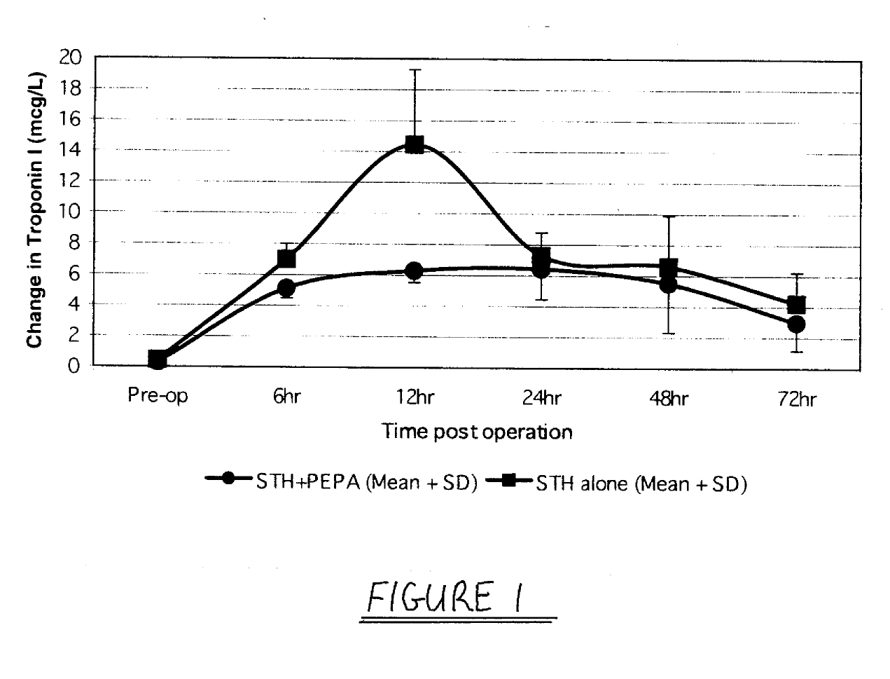

The results of the TnI analyses are indicated in Table 1 and FIG. 1 and in Table 2. The findings tabulated in Table 1 are depicted in FIG. 1.

TABLE 1
Changes from Pre-Bypass Baseline

| Time | STH + PEPA | STH |
|---|---|---|
| (hours) | [mean(SEM)] | [mean(SEM\|)] |
| Pre-bypass baseline | 0.25(0.12); n-37 | 0.03(0.01); n = 35 |
| 6 | 4.77(0.67); n = 37 | 6.64(1.06); n = 35 |
| 12 | 5.69(0.72); n = 36 | 13.66(4.84); n = 35 |
| 24 | 6.05(1.88); n = 36 | 10.87(4.43); n = 35 |
| 48 | 5.05(2.98); n = 35 | 6.44(3.38); n = 35 |
| 72 | 2.70(1.77); n = 32 | 4.24(2.12); n = 31 |

TABLE 2
Area Under Curve (“AUC”) from Pre-sternal Closure up
to 72 h in Changes from Pre-Bypass Baseline

| | STH + PEPA | STH |
|---|---|---|
| Total Patients | 37 | 35 |
| Mean(SD)/Median | 4.51(10.37)/2.38 | 7.68(18.65)/3.29 |
| Lower/Upper Quartile | 1.23/4.40 | 1.70/5.65 |
| Minimum/Maximum | −1.28/64.40 | 0.21/111.40 |

The results indicate that the use of PEPA markedly reduces the overall concentration of troponin I in the blood over the period studied in patients undergoing coronary artery bypass surgery. This trend is a clear indication that necrosis of heart muscle tissue is reduced during cardiac surgery as a result of the use of PEPA.

The Inventor is not aware of any existing treatment which reduces or inhibits necrosis of cardiac muscle tissue, for example, during cardiac surgery, following a recent cardiac event or before the onset of myocardial necrosis. Thus, embodiments of the invention represent a significant step forward in medical care. Further advantages of embodiments of the invention include:

an increased rate of survival of cardiac surgery or ACS;

reduced morbidity; and

a reduced need for surgical intervention or drug treatment for ACS.

It will be appreciated that the invention is not restricted to the details described above with reference to the preferred embodiments but that numerous modifications and variations can be made without departing from the spirit and scope of the invention as defined in the following claims.

1. A method of reducing or inhibiting necrosis of cardiac muscle tissue in a patient comprising administering to said patient a therapeutically effective amount of at least one compound having Formula (1): 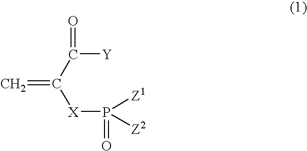
wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
and wherein:
R¹ to R⁶ are selected, independently, from the group consisting of H; alkyl; cycloalkyl; alkenyl; aryl; and aralkyl,
or a physiologically acceptable salt thereof.

2. The method according to claim 1 wherein the therapeutic amount provides a reduction in the peak change in troponin concentration in blood of a patient of at least 15% of the expected peak change in troponin concentration in blood.

3. The method according to claim 1 wherein the patient is undergoing a surgical intervention.

4. The method according to claim 3 wherein the patient is undergoing cardiac surgery on cardiopulmonary bypass.

5. The method according to claim 4 wherein observed level of necrosis of the cardiac muscle tissue at a given point within 72 hours after administration is less than the expected level of necrosis at said point if said compound(s) had not been administered.

6. The method according to claim 1 wherein the patient is at high risk of necrosis or cardiac muscle tissue.

7. The method according to claim 6 wherein the or each compound is administered before onset of necrosis.

8. The method according to claim 1 wherein the patient is suffering from acute coronary syndrome (“ACS”).

9. The method according to claim 8 wherein the or each compound is administered as soon as possible after diagnosis of ACS.

10. The method according to claim 9 wherein the or each compound is administered no more than 24 hours after a cardiac event.

11. The method according to claim 1 wherein the or at least one compound has Formula (1) in which X is O; Y is NR²R³; Z¹ is OH; and Z² is OH.

12. The method according to claim 10 wherein said compound has Formula (1) in which Y is NH₂.

13. A method of reducing necrosis of cardiac muscle tissue in a patient undergoing cardiac surgery comprising administering to said patient a therapeutically effective amount of at least one compound having Formula (1): 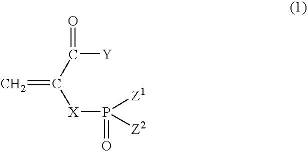
wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
and wherein:
R¹ to R⁶ are selected, independently, from the group consisting of H; alkyl; cycloalkyl; alkenyl; aryl; and aralkyl,
or a physiologically acceptable salt thereof.

14. The method according to claim 13 wherein the or each compound is administered via a cardioplegic solution prior to cardiopulmonary bypass.

15. A method of treatment of a patient suffering from ACS comprising administering to said patient a therapeutically effective amount of at least one compound having Formula (1): 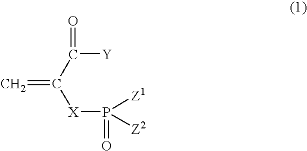
wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
and wherein:
R¹ to R⁶ are selected, independently, from the group consisting of H; alkyl; cycloalkyl; alkenyl; aryl; and aralkyl,
or a physiologically acceptable salt thereof.

16. A method of inhibiting necrosis of cardiac muscle tissue in a patient at high risk from necrosis of cardiac muscle tissue comprising administering to said patient a therapeutically effective amount of at least one compound having Formula (1): 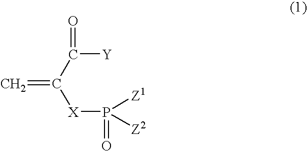
wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
and wherein:
R¹ to R⁶ are selected, independently, from the group consisting of H; alkyl; cycloalkyl; alkenyl; aryl; and aralkyl,
or a physiologically acceptable salt thereof.

17. A method of limiting troponin levels in blood of a patient comprising administering a therapeutically effective amount of at least one compound having Formula (1): 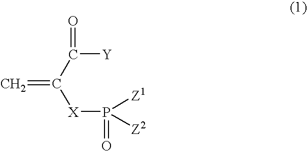
wherein:
X is selected from the group consisting of O; and NH;
Y is selected from the group consisting of OH; OR¹; and NR²R³;
Z¹ and Z² are selected, independently, from the group consisting of OH; OR⁴; and NR⁵R⁶;
and wherein:
R¹ to R⁶ are selected, independently, from the group consisting of H; alkyl; cycloalkyl; alkenyl; aryl; and aralkyl,
or a physiologically acceptable salt thereof.
